(19) United States
(12) Patent Application Publication
(10) Pub. No.: US 2016/0199661 A1
(43) Pub. Date: Jul. 14, 2016

(54) CANCER CELL MEMBRANE DEPOLARIZATION

(71) Applicant: BOSTON SCIENTIFIC SCIMED, INC., Maple Grove, MN (US)

(72) Inventors: Martin R. Willard, Burnsville, MN (US); Derek C. Sutermeister, Ham Lake, MN (US)

(21) Appl. No.: 14/993,735

(22) Filed: Jan. 12, 2016

Related U.S. Application Data
(60) Provisional application No. 62/102,810, filed on Jan. 13, 2015.

(51) Int. Cl. A61N 1/40

(52) U.S. Cl. CPC A61N 1/40

(57) ABSTRACT

Systems, methods, and devices for treating cancer can produce field potentials of between 20 mV and 70 mV. In some cases, systems, methods, and devices provided herein can include a plurality of particles. The particles can include at least a first material and at least a second material, which can have electrode potentials configured to form a galvanic couple in the location of cancerous tissue to form a field potential adapted to preferentially target cancerous tissue over healthy tissue.

### CROSS-REFERENCE TO RELATED APPLICATIONS

This application claims priority under 35 U.S.C. §119 to U.S. Provisional Application Ser. No. 62/102,810, filed Jan. 13, 2015, the entirety of which is incorporated herein by reference.

### TECHNICAL FIELD

This disclosure relates to systems, methods, and devices for membrane depolarization in cancerous cells. For example, systems, methods, and devices provided herein can be used to ablate tumors.

### BACKGROUND

Cancer treatments, such as tumor removal surgeries, radiation therapy, and chemotherapy, generally seek to remove or kill cancerous cells while leaving healthy cells intact. Non-hematological cancers can be cured if entirely removed by surgery, but this is not always possible. Additionally, a single cancer cell, invisible to the naked eye, can regrow into a new tumor, thus ensuring that the cancer is entirely removed can require the removal of significant amounts of healthy tissue. Surrounding healthy tissue may be too important to remove.

Radiation therapy can focus ionizing radiation via external beam radiotherapy (EBRT) or internally via brachytherapy at the location of cancerous cells to kill cancer cells and shrink tumors. In addition to damaging the genetic material in the cancerous cells, however, radiation therapy can also damage healthy cells in and around the targeted location.

Chemotherapy drugs interfere with cell division in various possible ways, e.g. with the duplication of DNA or the separation of newly formed chromosomes. Because most forms of chemotherapy target all rapidly dividing cells, not just cancerous cells, chemotherapy drugs have the potential to harm healthy tissue, especially those tissues that have a high replacement rate (e.g. intestinal lining).

Immunotherapy, which can include various therapeutic strategies designed to induce the patient's own immune system to fight the tumor, is promising, but can also result in severe side effects. Newer targeted therapies include monoclonal antibody therapy, photodynamic therapy, and molecularly targeted therapy.

The choice of therapy depends upon the location and grade of the tumor and the stage of the disease, as well as the general state of the patient, and a treatment strategy can sometimes include the use of multiple therapies. Because no treatment is perfect, there is a continuing need to develop additional cancer treatments.

### SUMMARY

Systems, methods, and devices provided herein can be used to depolarize cell membranes in cancerous cells. Cell membrane depolarization can lead to cell necrosis. Systems, methods, and devices provided herein can apply a field potential to cancerous cells to depolarize cancerous cells. In some cases, systems, methods, and devices provided herein can provide a field potential a cancerous tumor and surrounding healthy tissues to depolarize cancerous cells without depolarizing a majority of surrounding healthy cells. In some cases, an applied field potential can be between 20 mV and 70 mV. In some cases, an applied field potential can be between 25 mV and 65 mV, between 30 mV and 60 mV, between 35 mV and 55 mV, or between 40 mV and 50 mV. In some cases, an applied field potential can be between 25 mV and 70 mV, between 30 mV and 70 mV, between 35 mV and 70 mV, between 40 mV and 70 mV, between 45 mV and 70 mV, between 50 mV and 70 mV, between 55 mV and 70 mV, between 60 mV and 70 mV, or between 65 mV and 70 mV. In some cases, an applied field potential can be between 20 mV and 65 mV, between 20 mV and 60 mV, between 20 mV and 55 mV, between 20 mV and 50 mV, between 20 mV and 45 mV, between 20 mV and 40 mV, between 20 mV and 35 mV, between 20 mV and 30 mV, or between 20 mV and 25 mV. In some cases, systems, methods, and devices provided herein can be combined with additional cancer therapies to treat a cancerous tumor. For example, devices delivered to a location including cancerous cells can both create a field potential and deliver chemotherapy drugs.

In some aspects, systems, methods, and devices provided herein can create a field potential by creating a galvanic couple. In some cases, systems, methods, and devices provided herein can incorporate at least two materials that have electrode potentials that differ by between 20 mV and 70 mV to create a desired field potential. For example, a first material can include calcium having an electrode potential of about −2870 mV and a second material can include barium having an electrode potential of about −2800 mV to produce a field potential of about 70 mV. In some cases, zinc (electrode potential of about −760 mV) and chromium (electrode potential of about −740 mV) can be combined to produce a field potential of about 20 mV. In some cases, cobalt (electrode potential of about −280 mV) and nickel (electrode potential of about −240 mV) can be combined to produce a field potential of about 40 mV.

In some aspects, systems, methods, and devices provided herein can incorporate particles of two or more different materials having electrode potentials adapted to create a desired field potential. For example, particles can be directly injected into a tumor and surrounding tissues or administered during open surgery to create a desired field potential in and/or around the tumor. In some cases, systems, methods, and devices provided herein can incorporate microparticles including the two or more materials. For example, microparticles provided herein can have an average particle diameter of between 1 μm and 1000 μm. In some cases, systems, methods, and devices provided herein can incorporate nanoparticles including the two or more materials. For example, nanoparticles provided herein can have an average particle diameter of between 1 nm and 1 μm. In some cases, particles provided herein can have an average particle diameter of between 1 nm and 1000 μm, between 10 nm and 500 μm, between 50 nm and 100 μm, between 100 nm and 50 μm, between 500 nm and 10 μm, or between 1 μm and 5 μm. In some cases, particles provided herein can have an average particle diameter of between 1 nm and 500 μm, between 1 nm and 100 μm, between 1 nm and 10 μm, between 1 nm and 5 μm, between 1 nm and 500 nm, between 1 nm and 100 nm, between 1 nm and 10 nm, or between 1 nm and 5 nm. In some cases, particles provided herein can have an average particle diameter of between 10 nm and 1000 μm, between 100 nm and 1000 μm, between 500 nm and 1000 μm, between 10 μm and 1000 μm, between 100 μm and 1000 μm, or between 500 μm and 1000 μm.

In some aspects, systems, methods, and devices provided here can include a first set of particles comprising a first material and a second set of particles comprising a second material. In some cases, the first set of particles is substantially free of the second material and the second set of particles is substantially free of the first material. In some cases, the first set of particles can consist essentially of the first material and the second set of particles can consist essentially of the second material. In some cases, the first set of particles can consist of the first material and the second set of particles can consist of the second material. In some cases, each particle can include both the first and the second material with a salt bridge there between. In some cases, a salt bridge can include a hydrophobic polymer or fiber.

In some aspects, systems, methods, and devices provided herein can include a plurality of particles suspended in a carrier. In some cases, the carrier can be a gel carrier. In some cases, the carrier can be water or an aqueous solution. In some cases, the carrier can be saline solution. In some cases, the carrier can be conductive. In some cases, the carrier can be nonconductive. For example, a nonconductive carrier can mix with bodily tissues to create a conductive electrolyte to create a galvanic couple between the particles.

In some aspects, systems, methods, and devices provided herein can be adapted to mix and/or deliver particles to a location including cancerous cells. In some cases, systems, methods, and devices can include an injector containing particles (and optional one or more carriers). In some cases, an injector can isolate a first composition including a first group of particles including a first material from a second composition including a second group of particles including a second material to prevent the formation of a galvanic couple between the first and second materials prior to injection into body tissue. In some cases, the injector can mix the first composition and the second composition prior to or during the injection of the first and second compositions into body tissue. In some cases, an injector provided herein can inject particles of one or more materials. In some cases, an injector provided herein accelerate particles towards body tissues to imbed particles directly into the body tissue. In some cases, the particles may be provided dry or in a non-hydrophilic carrier such as an oil. In some cases, particles may be prepped using a hydrophilic carrier for injection. In some cases, particles may be placed dry or with liquid preparation as part of a surgical resection case.

The details of one or more embodiments are set forth in the accompanying description below. Other features, objects, and advantages will be apparent from the description and from the claims.

### DETAILED DESCRIPTION

Systems, methods, and devices provided herein can be used to depolarize cell membranes in cancerous cells. Cell membrane depolarization can lead to cell necrosis. Systems, methods, and devices provided herein can provide a field potential that preferentially kills tumor cells over healthy cells. In some cases, a field potential provided by systems, methods, and devices provided herein can depolarize a majority of tumor cells, but leave a majority of healthy cells polarized.

### Cell Membrane Depolarization and Necrosis

Cell membrane depolarization is a positive-going change in a cell's membrane potential, making it more positive, or less negative, and thereby removing the polarity that arises from the accumulation of negative charges on the inner membrane and positive charges on the outer membrane of the cell. If, for example, a cell has a resting potential of −70 mV, once the membrane potential changes to −50 mV, then the cell has been depolarized. Depolarization can typically be caused by an influx of cations, e.g. Na⁺ through Na⁺ channels, or Ca²⁺ through Ca²⁺ channels. These channels, also known as voltage-dependent ion channels, open when an action potential begins, or at the threshold potential. On the other hand, efflux of K⁻ through K⁺ channels inhibits depolarization, as does influx of Cl⁻ (an anion) through Cl⁻ channels. If a cell has K⁺ or Cl⁻ currents at rest, then inhibition of those currents will also result in a depolarization.

Systems, methods, and devices provided herein can depolarize cell membranes by applying a field potential. The membrane depolarization potential of cancer cells are typically lower than that of healthy cells. Systems, methods, and devices provided here can immerse cancerous tissues and surrounding healthy tissue in a field potential of greater than 20 mV and less than 70 mV to trigger a preferential depolarization of cancerous cells over healthy cells. In some cases, field potential provided herein can depolarize mitochondria in cancerous cells preferentially over mitochondria in healthy cells. In some cases, depolarization in mitochondria can lead to ATP depletion, followed by a disturbance of ion homeostasis, cellular Ca2+ overloading, and finally cellular necrosis. This is primarily due to the increased metabolic rate of tumor cells compared to healthy (normal) tissue.

### Field Potentials

Systems, methods, and devices provided herein include a variety of methods of creating and applying field potentials to body tissues, particularly body tissues including cancerous cells. In some cases, systems, methods, and devices provided herein can combine two or more materials to create a galvanic couple. In some cases, the two or more materials can include metals. Each material can have an electrode potential, and the difference between the electrode potentials represents the field potential that can be produced by that combination of materials. Table 1 below shows exemplary combinations of elements that can be used in systems, methods, and devices provided herein to create a field potential of between 20 mV and 70 mV.

TABLE 1

| | | | Combination |
|---|---|---|---|
| Combination | Element | Electrode Potential (Volts) | Potential (mV) |
| 1 | Calcium | −2.87 | 70 |
| | Barium | −2.80 | |
| 2 | Zinc | −0.76 | 20 |
| | Chromium | −0.74 | |
| 3 | Iron | −0.44 | 40 |
| | Cadmium | −0.40 | |
| 4 | Cobalt | −0.28 | 40 |
| | Nickel | −0.24 | |
| 5 | Silver | 0.80 | 50 |
| | Mercury | 0.85 | |

In some cases, an applied field potential can be between 20 mV and 70 mV. In some cases, an applied field potential can be between 25 mV and 65 mV, between 30 mV and 60 mV, between 35 mV and 55 mV, or between 40 mV and 50 mV. In some cases, an applied field potential can be between 25 mV and 70 mV, between 30 mV and 70 mV, between 35 mV and 70 mV, between 40 mV and 70 mV, between 45 mV and 70 mV, between 50 mV and 70 mV, between 55 mV and 70 mV, between 60 mV and 70 mV, or between 65 mV and 70 mV. In some cases, an applied field potential can be between 20 mV and 65 mV, between 20 mV and 60 mV, between 20 mV and 55 mV, between 20 mV and 50 mV, between 20 mV and 45 mV, between 20 mV and 40 mV, between 20 mV and 35 mV, between 20 mV and 30 mV, or between 20 mV and 25 mV.

### Particles

In some cases, systems, methods, and devices provided herein can produce field potentials using particles, which can be delivered to the body tissue. In some cases, a first group of particles can include a first material and a second group of particles can include a second material where the electrode potential difference between the two materials is between 20 mV and 70 mV.

Particles used in systems, methods, and devices provided herein can have any suitable size and/or shapes. In some cases, a composition used in systems, methods, and devices provided herein can include a plurality of nanoparticles and/or microparticles including two or more materials adapted to produce a field potential of between 20 mV and 70 mV. For example, a composition provided herein can include zinc nanoparticles and chromium nanoparticles. Particles provided herein can be amorphous, partial-crystalline, or crystalline. In some cases, particles provided herein can include alloys.

Particles provided herein can be combined with drugs and/or other therapeutics. For example, particles provided herein can have a coating of a chemotherapy drug. In some cases, particles provided herein can include radioactive isotopes adapted to provide radioactive therapy to body tissue including cancerous cells. In some cases, particles provided herein can be used during or after an operation used to surgically remove cancerous tissue. For example, particles provided herein can be accelerated and implanted into body tissue surrounding an area where a tumor has been removed. In some cases, particles provided herein can be used to heat a tumor while providing the field potential. In some cases, particles can be heated using the Curie Temp particle technique. In some cases, the particles can be ferromagnetic in order to use the Curie Temp particle technique.

Particles provided herein can be biodegradable due to the formation of the galvanic couple. The time period for complete degradation can be a few weeks to several months, and the products are non-toxic and do not disturb cell level functions. The degradation products are excreted out, for example. Particles provided herein can be porous or nonporous. The porosity of the particles can impact the degradation rate.

Particles provided herein can be included in a carrier. A carrier provided herein can include water, aqueous solutions (e.g., saline solution), and gels. In some cases, individual particles or groups of particles can be included in a matrix of a carrier. The carrier can degrade or disperse when the particles are delivered to the body tissue. In some cases, a carrier can be conductive. In some cases, a carrier can be non-conductive and thus preserve the particles prior to implantation.

Particles provided herein can be made using any suitable process. In some cases, particles used in systems, methods, and devices provided herein can be made using sputtering-based gas phase condensation, mechanical alloying, electro-deposition, and/or chemical methods. For example, nanoparticles used in systems, methods, and devices provided herein can be made using sputtering-based nanoparticle fabrication system, where a high negative voltage is applied to a tube target and Ar sputtering gas is injected through the tube target hole. The high negative voltage ionize the Ar gas to generate Ar+ ions which will be accelerated to hit the inside wall of the target to knock out the atoms. Then the knocked out atoms are carried out of the target to form high density atoms. At high pressure environment, the atom gas condenses to form nanoparticles including atoms carried from the target. Sputtering pressure can be 500 mTorr to 2 Torr and sputtering power can be 100 W to 400 W. In some cases, nanoparticles of two of more different materials can be sputtered directly into body tissue. In some cases, nanoparticles of two of more different materials can be deposited onto a surface, transferred into a carrier (e.g., water, saline solution, or gel), and then delivered to body tissue.

Particles used in systems, methods, and devices provided here can in some cases be functionalized to allow the nanoparticles to be suspended in a carrier or water soluble. In some cases, polyethylene glycol (PEG) can be coated onto particles provided herein. Polymers instead of PEG, such as glucose, biodegradable thermal sensitive POEG, can also be used for surface functionalization. Besides APTES modification, incorporation of —CHO group onto the surface can be realized through EDC/sulfNH2. Covalent bonds are formed in the presence of —CHO group. In some cases, particles provided herein can be functionalized with specific targeting groups for specific types of cells or tissues.

### Particle Delivery

Particles including two or more materials adapted to produce a field potential of between 20 mV and 70 mV used in systems, methods, and devices provided herein can be delivered to a location including cancerous cells using any suitable method. In some cases, particles producing a desired field potential of between 20 mV and 70 mV can be injected directly into a location including cancerous cells (e.g., a tumor). In some cases, particles producing a desired field potential of between 20 mV and 70 mV can be accelerated and implanted into a target location. For example, during surgery, particles provided herein can be sputtered an area suspected of including cancerous cells (e.g., tissue adjacent to a removed tumor). In some cases, particles can be functionalized with specific targeting groups for specific types of cells or tissue and can be injected into a patient's blood stream.

In some cases, systems, methods, and devices provided herein can include an injector. In some cases, an injector can retain a plurality of particles adapted to produce a filed potential of between 20 mV and 70 mV when injected. In some cases, a first group of particles including a first material can be isolated from a second group of particles including a second material and the injector can be adapted to mix the first group of particles with the second group of particles prior to or during the injection of particles out of the injector. In some cases, an injection apparatus provided herein can including a particle mixing and/or preparation chamber. In some cases, an injection apparatus provided herein can including a particle injection volume and/or concentration controller. In some cases, an injection apparatus provided herein can be adapted to automatically dispense particles based on tissue durometer, vascularization, and/or density. In some cases, an injection apparatus provided herein can include a single delivery port for multiple particles to be delivered there through. In some cases, an injection apparatus provided herein can be adapted to mix different types of particles for injection through a single delivery port. In some cases, an injection apparatus provided herein can be adapted to delivery different types of particles through a single delivery port sequentially. In some cases, an injection apparatus provided herein can include multiple delivery ports for simultaneous or sequential delivery of different types of particles. In some cases, an injection apparatus provided herein can mix drugs, chemo agents, and/or other treatments into a solution mixed with particles.

All publications, patent applications, patents, and other references mentioned herein are incorporated by reference herein in their entirety.

Still further embodiments are within the scope of the following claims.

What is claimed is:

1. A method of treating cancerous tissue comprising generating a field potential in a location of cancerous tissue, the field potential being between 20 mV and 70 mV.

2. The method of claim 1, wherein the field potential is generated by creating a galvanic couple between at least two different materials in the location.

3. The method of claim 2, wherein a first material comprises calcium and a second material comprises barium.

4. The method of claim 2, wherein a first material comprises zinc and a second material comprises chromium.

5. The method of claim 2, wherein a first material comprises cobalt and a second material comprises nickel.

6. The method of claim 1, wherein the field potential created by delivering a plurality of particles to the location.

7. The method of claim 6, wherein the particles comprise microparticles, nanoparticles, or a combination thereof.

8. The method of claim 6, wherein the particles comprise a chemotherapy drug, a radioactive isotope, or a combination thereof.

9. The method of claim 1, wherein the method further comprises surgically removing cancerous tissue, delivering chemotherapy drugs to the cancerous tissue, radiation therapy, immunotherapy, or a combination thereof.

10. The method of claim 6, wherein the method further comprises ferromagnetic particle heating of the cancerous tissue.

11. A system for treating cancer comprising a plurality of particles, the plurality of particles comprising a first material and a second material, the first material and the second material each having electrode potentials configured to form a field potential of between 20 mV and 70 mV.

12. The system of claim 11, wherein the particles comprise microparticles, nanoparticles, or a combination thereof.

13. The system of claim 11, wherein the particles comprise a first set of particles comprising the first material and a second set of particles comprising the second material.

14. The system of claim 13, the system comprising an injection apparatus adapted to combine the first set of particles with the second set of particles and introduce the particles into body tissue.

15. The system of claim 11, wherein the first material comprises calcium and the second material comprises barium.

16. The system of claim 11, wherein the first material comprises zinc and the second material comprises chromium.

17. The system of claim 11, wherein the first material comprises cobalt and the second material comprises nickel.

18. The system of claim 11, further comprising a carrier, the particles being suspended in the carrier.

19. The system of claim 11, further comprising a chemotherapy drug, a radioactive isotope, or a combination thereof.

20. An implantable medical device comprising a first material and at least a second material, the first material and the second material each having electrode potentials configured to form a field potential of between 20 mV and 70 mV, the implantable medical device being adapted to be implanted into cancerous tissue.
